(19) United States
(12) Patent Application Publication
(10) Pub. No.: US 2020/0197212 A1
(43) Pub. Date: Jun. 25, 2020

(54) Three-Dimensional Image Capturing System

(71) Applicants: Curtis Bryan Carlson, Hickman, NE (US); Scott Strasburger, Lincoln, NE (US); Doak Ostergard, Lincoln, NE (US)

(72) Inventors: Curtis Bryan Carlson, Hickman, NE (US); Scott Strasburger, Lincoln, NE (US); Doak Ostergard, Lincoln, NE (US)

(21) Appl. No.: 16/228,062

(22) Filed: Dec. 20, 2018

(51) Int. Cl. A61F 5/14; A61B 5/00; A61B 5/107; A61B 5/103; A61B 90/00; G06F 30/10; G16H 30/20; G06T 19/20

(52) U.S. Cl. CPC A61F 5/14; A61B 5/0079; A61B 5/70; A61B 5/1074; A61B 5/1079; A61B 5/1036; G06T 2210/41; A61B 5/1077; G06F 30/10; G16H 30/20; G06T 19/20; A61B 2090/3937; A61B 90/39

(57) ABSTRACT

This disclosure relates to the technique for acquiring a three-dimensional image of the human body for the purpose of manufacturing customized orthopedic braces.

### FIELD OF THE INVENTION

This present invention relates to the field of orthopedic bracing devices and methods, collectively a system, for creating fully-customized orthoses, which involves acquiring a plurality of three-dimensional images in a weight-bearing position of the human body to form an anatomical model and manipulating the said model to form a fully-customized orthoses.

### BACKGROUND OF THE INVENTION

Orthoses, or the correction of disorders of the limbs or spine by use of braces and other devices to correct alignment or provide support, are a necessity for a variety of patients. Some common types of orthoses are foot orthoses, ankle orthoses, ankle and foot orthoses (AFO), knee orthoses, back orthoses, and wrist orthoses. AFOs are orthoses specifically for the lower leg which enclose the ankle and foot below the knee. AFOs can be made of many different materials to maintain the desired level of control and stabilize the ankle. Common problems relieved by AFOs are drop-foot, fractures, ankle instability, and other post-operative indications, to name a few.

Once the need for an orthosis is determined, a physician will then determine the need for either a custom or prefabricated version. Both custom and prefabricated orthoses are beneficial for recovery, rehabilitation, and protection against reoccurring injury. Prefabricated orthoses are typically options for less severe injuries, short-term use, or to keep costs at a minimum. These orthoses are usually fitted by taking circumferential and/or length measurements and choosing the closest corresponding standard size: small, medium, or large, for example.

Custom orthopedic orthoses offer the benefits of individualization in contrast to the prefabricated, over-the-counter, “closest fit” orthoses by reducing risk and reoccurrence of injuries through the customization of support features. Fit customization is an important design factor for obtaining optimal function. The main advantages for selecting a custom orthosis is achieving a proper fit, preventing migration or exaggerated injury due to faulty support, as well as helping to maintain appropriate centralized pressure due to the contoured fit reducing direct force on an area or the skin. Custom orthoses can also provide added stabilization and balance again due to the centralized pressure maintained by the contoured surface. Custom orthoses are also crucial for those with unique anatomical features, abnormalities, and deformities which tend not to fit into standard sizes. These types of orthoses are typically options for more severe injuries, athletes competing in high-impact athletics, and long-term use.

Once it is determined that the patient needs an orthosis, methods for making custom orthoses vary and include casting, measurement systems, scanners, and recently electronic applications on portable computing devices used to capture a plurality of images of the affected anatomical portion and when meshed together create a digital impression or mold of the body.

Creating a plaster impression of the foot, ankle, or lower leg has traditionally been the method for practitioners who make custom orthopedic orthoses for their patients. These labor-intensive casting techniques can be time-consuming, messy, difficult to store, material intensive, uncomfortable for the patient, and costly for both the physician and the patient. Most concerning, however, is that the plaster method used to create custom orthopedic orthoses do not produce a custom fit. A cast impression cannot position the foot, ankle, or lower leg in a weight-bearing position, leading to unreliable measurements and a poor fit for the patient.

An additional process used in the fabrication of creating a more custom AFO is referred to as heat molding. This method consists of several heavily-involved steps including molding, vacuum-heat-forming, and fitting. In addition to being laborious, an additional disadvantage to this method is that the practitioner is not able to manipulate the orthosis to position the foot for function and pain relief. The difficulty occurs when trying to correct an anatomical portion which cannot be done in a non-weight-bearing position.

Laser scanners, used to capture a three-dimensional, digital plantar image of the foot, combined with computer-aided design (CAD) produce custom orthotics, orthotic molds, or soft insoles that provide cost and time advantages for both the patient and practitioner. However, these only produce a topographical image, illustrating the hills and valleys of the foot, leaving the dimensions of the ankle and lower leg a mystery, which are essential to achieving a proper fit.

Another technique captures multiple two-dimensional, sequential images of the foot, compressing those images to create a three-dimensional model. Difficulties with this method emerge when the practitioner attempts to translate the two-dimensional, sequential images to a final orthosis in the area of anatomical accuracy.

In more recent years, technology has permitted the use of three-dimensional foot scanning, CAD, and computer-aided manufacturing in the fabrication of foot molds and custom foot orthotics. Three-dimensional printing has proven successful in the fabrication of an AFO. More specifically, methods have been disclosed for digitally “best fitting” a user to a variety of wearable variations. For example, in the U.S. Pat. No. 9,460,557 and in the area of footwear, images of a user's anatomical portion are captured from a plurality of angles using a mobile camera, creating a three-dimensional model of the user's anatomical portion from the images, and selecting a “best-fit” based on the three-dimensional model. This method is used to provide convenience and efficiency when selecting a prefabricated orthosis. This scanning method utilizes templates, or predetermined measurements, based on a patient's gender, age, or weight. In this process, the practitioner first forms a set of deformable models of the anatomical portion such as the face, hand, or foot. The deformable three-dimensional master model is subsequently fit to the user's body through reference points. However, this method, using templates, falls short of creating a fully-customized orthosis.

Physicians would benefit from an image-capturing device that utilizes a method that would provide a quick, fully-customized collection of three-dimensional images of the foot, ankle, and leg in a true weight-bearing position that when meshed together create a carbon copy of the patient's anatomical portion used to produce a three-dimensional, custom orthopedic orthosis in a short amount of time, with in-office convenience, without the mess, resulting in more precise treatment.

The process of capturing, meshing, and uploading a three-dimensional model of an anatomical portion can take a matter of minutes. This essentially takes the custom impression fitting and production process from weeks to a matter of hours. Another advantage of this three-dimensional, image-capturing process is that it reduces the waste typically associated with the plaster and foam molding methods using only the required material.

### BRIEF DRAWING DESCRIPTIONS

FIG. 1 Illustrates an orthographic view of the image-capturing platform.

FIG. 2 Illustrates a perspective view of the image-capturing platform with a monopod hosting an image-capturing device.

FIG. 3 Illustrates an orthographic top view of platform base of the image-capturing platform with fiducial identification markers and the interior/exterior platform base lighting.

FIG. 4 Illustrates a perspective view of the image-capturing platform with the transparent top panel and mirror removed from the platform base.

FIG. 5 Illustrates a perspective view of a mobile computing device, here a tablet, hosting a three-dimensional image capturing device.

FIG. 6 Illustrates a perspective view of a patient standing on the three-dimensional image-capturing device with one foot in a weight-bearing position, the other on the footrest, and the plantar region of the foot reflected in the mirror, located inside the platform base, as well as an image-capture device attached to the monopod, maneuvered by a practitioner.

FIG. 7 Illustrates a perspective view of a patient standing on an image-capturing platform with one foot in a weight-bearing position, the other on the footrest, and the plantar region of the foot reflected in the mirror, located inside the platform base, as well as an image-capture device detached from the monopod and hand-held by a practitioner.

FIG. 8 Illustrates a perspective view of a three-dimensional, fully-customized anatomical model of the foot, ankle, and lower leg of a patient in a weight-bearing position.

FIG. 9 Illustrates a perspective view of a manipulated, three-dimensional, fully-customized anatomical model of the foot, ankle, and lower leg of a patient in a weight-bearing position with the plantar region re-reflected and aligned with upper portion of the foot and meshed together.

FIG. 10 Illustrates a perspective view of a collection of different three-dimensional, fully-customized, meshed images of the foot, ankle, and lower leg of a patient in a weight-bearing position, including a CAD model and a CAD model restructured as a printable lattice model.

FIG. 11 Illustrates a perspective view of a three-dimensional model of the foot, ankle, and lower leg, further edited by using a digital stencil or template to remove the unnecessary portions of the model.

FIG. 12 Illustrates a perspective view of the axis of rotation of the ankle, as well as the appropriate location of a cam, or joint, based on patient-specific anatomic landmarks.

FIG. 13 Illustrates a perspective view of a fully-customized, orthopedic orthosis based on the image-capture system, printed and assembled with the cam inserted at the ankle joint line.

FIG. 14 Illustrates an algorithm used to create and manipulate image data to produce a fully-customized, orthopedic orthosis.

### SUMMARY OF THE INVENTION

A device and method, collectively a system, which captures three-dimensional images of the foot, ankle, and lower leg in a true weight-bearing position that when meshed together create an accurate, three-dimensional, anatomical model used to create a fully-customized orthopedic orthosis is disclosed.

In one embodiment, the practitioner positions the patient on the image-capturing platform, comprised of a platform base, ring-like frame, transparent top panel, bottom panel, support bar, armrest, and footrest. In another embodiment, the platform base is an oval or a square. The patient places the affected foot on the transparent top panel of the platform base and rests the non-affected foot on the footrest. A flat mirror, affixed to the interior of the platform base, located below the transparent top panel of the platform base, provides the practitioner a reflection of the plantar region of the foot in a weight-bearing position and the optimal angle to capture three-dimensional images of the foot. In another embodiment, rim lighting lines the interior and exterior of the top of the platform base creating optimal lighting for consistent three-dimensional imaging.

In one embodiment a guide track, which provides negative space for a guide roller used to host a monopod, is affixed to the outer rim and encircles the platform base. The monopod, used to host an image-capturing device, can travel up to 360° around the patient's anatomical portion. In an exemplarily embodiment, the image-capturing device is hand-held, or removed from the monopod, and the practitioner manually orbits around the patient to capture any anatomical portion of the body.

Three-dimensional imaging is initiated by launching a user-interface and user-experience (UI/UX) application. The image-capturing device continuously obtains images of the anatomical portion of interest by collecting data from a plurality of angles and points. Fiducial identification markers are located at equally-spaced locations around the circumference of the platform base used to orient the images captured.

The UI/UX application begins to work with the data to form a merged anatomical model by identifying the points of the plantar region along with the points of the upper portion of the foot, ankle, and lower leg. It then re-reflects the points of the reflected plantar image of the foot and meshes them with the points of the upper portion of the foot, ankle, and lower leg, creating a full weight-bearing, three-dimensional model.

Once the full anatomical portion has been captured, an algorithm, or a finite sequence of steps used to solve a problem, engages by analyzing and comparing the images to each other, trimming and removing any residual reflections or unwanted visual elements or interference, isolating the data, cleaning and smoothing the surfaces of the meshed images into a smooth, three-dimensional, customized, polygonal, mesh shell of the patient's anatomical portion.

The three-dimensional, polygonal, mesh shell is then converted into a workable CAD model. The model will be used as a reference to customize an orthosis. In another embodiment, the CAD model can be restructured as a three-dimensional, printable, lattice structure also used as a reference to customize an orthosis, which allows focused support at various anatomical points.

The practitioner is then able to modify and manipulate the image within the UI/UX application to meet requirements such as desired positioning. The model is then further edited by placing a stencil or template onto the model, removing the unnecessary portions of the model, as well as increasing the exterior thickness and subtracting the interior section to achieve minimal tolerance to the anatomical topography, ultimately resulting in highly-customized components.

A cam, or joint, based on patient-specific anatomic landmarks is then digitally inserted at the joint line, or axis of rotation, providing the user anatomic function and protection in contrast to only providing restricted motion.

The completed files are then sent to a three-dimensional printer or in another embodiment to a computer-numerical-control (CNC) device. The fully-customized, orthopedic orthosis is printed and assembled.

### DETAILED DESCRIPTION OF THE INVENTION

Embodiments are described more fully below with reference to the accompanying figures which illustrate specific exemplary embodiments. These embodiments are disclosed in sufficient detail to enable those skilled in the art to practice the invention. However, embodiments may be implemented in many different forms and should not be construed as being limited to the embodiments set forth. The following detailed description is, therefore, not to be taken in a limiting sense in that the scope of the present invention is defined by the claims.

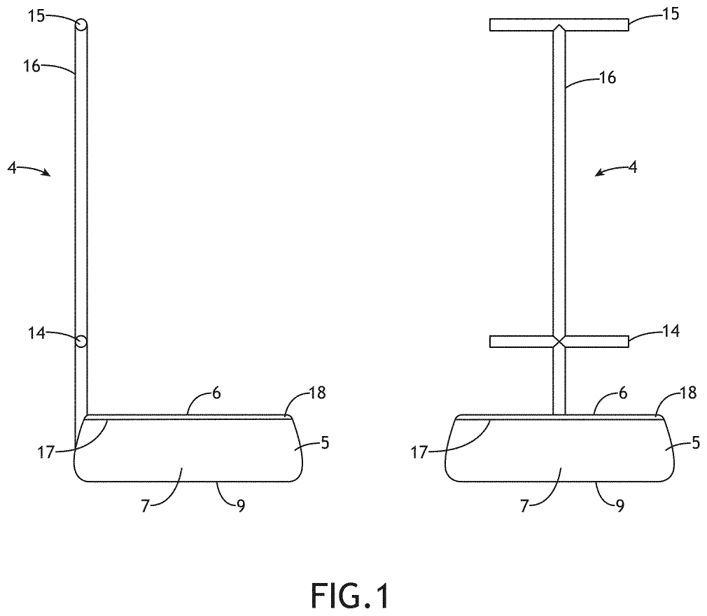

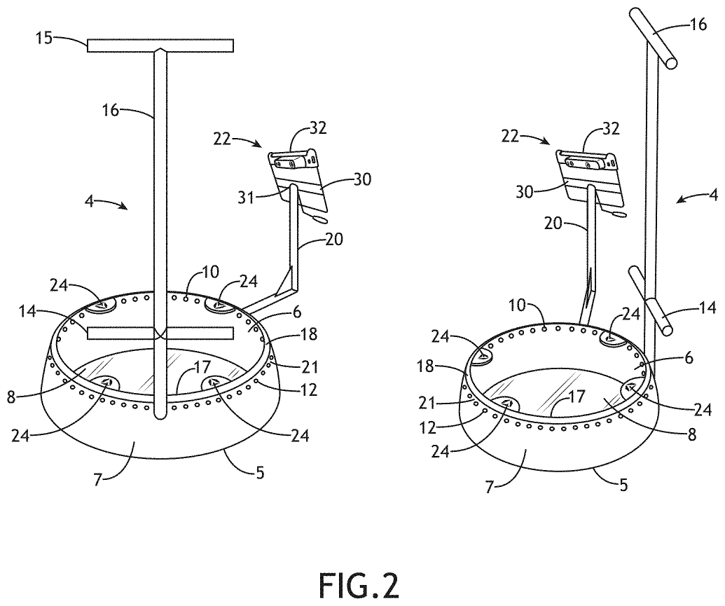

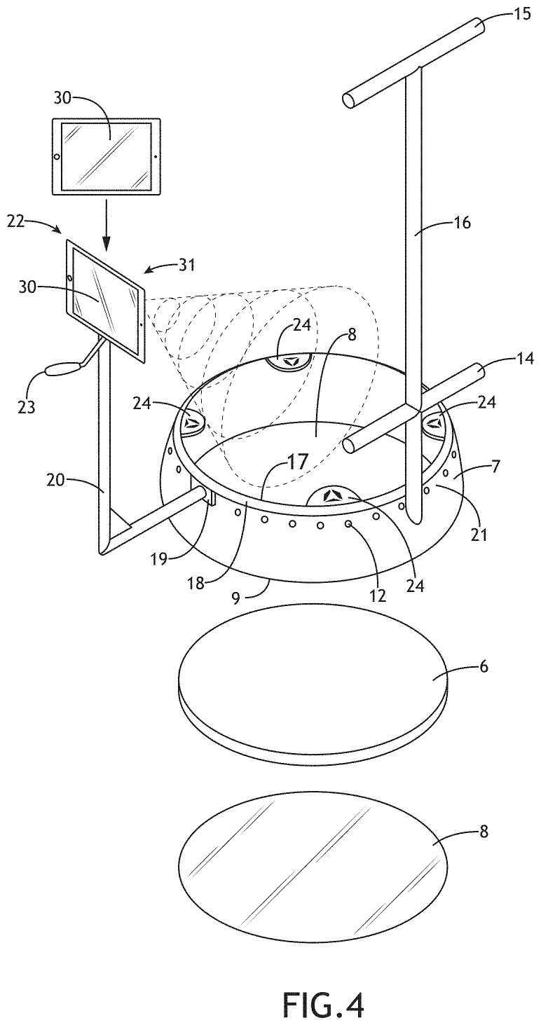

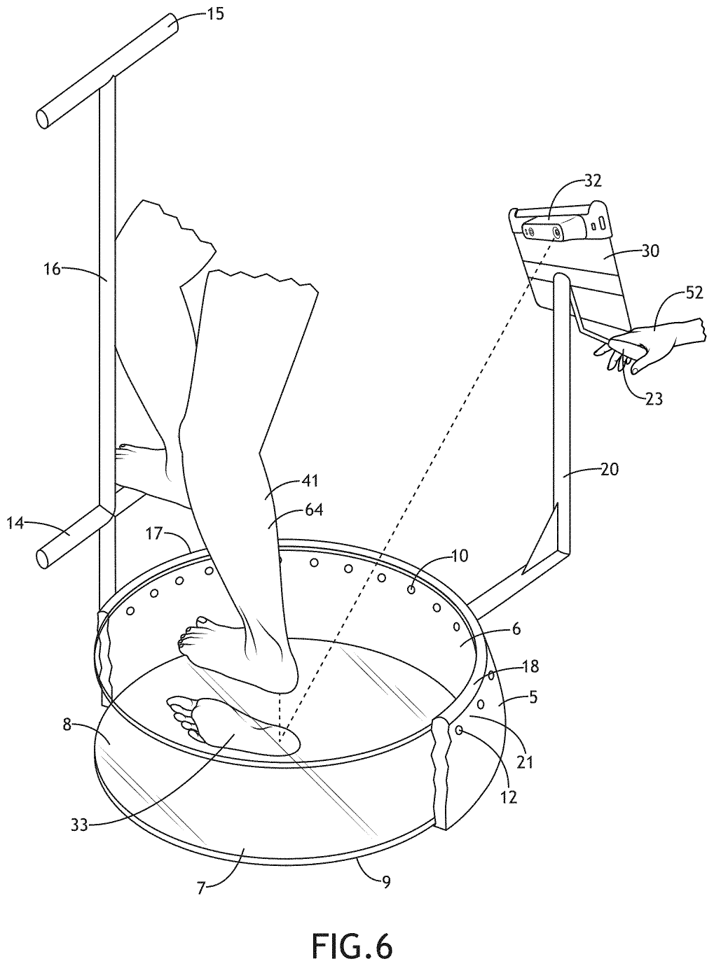

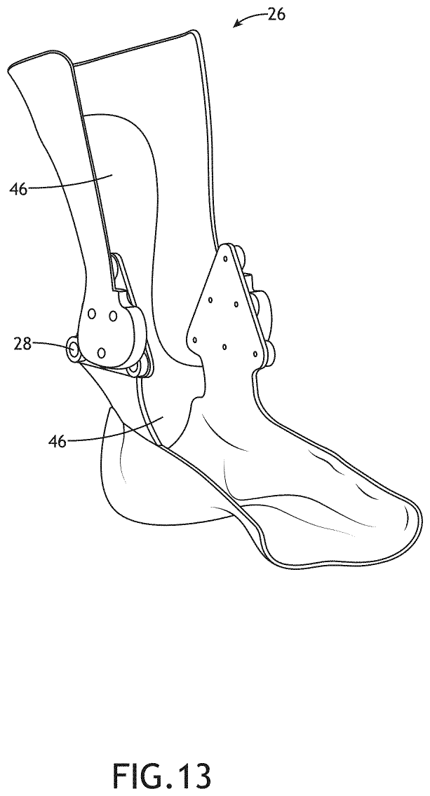

Numeral 4 refers to an image-capturing platform, as seen in FIG. 1, designed to create a fully-customized, orthopedic orthosis 26 for a patient, as seen in FIG. 13. The image-capturing platform 4 includes a platform base 5, with a ring-like frame 7, a bottom panel 9, and finally enclosed by a transparent top panel 6. Inside the platform base 5, a mirror 8, mimics the shape of the platform base 5, as seen in FIG. 4, providing the practitioner 52 a reflection of the plantar image of the foot 33 which provides a weight-bearing position for image capturing as seen in FIG. 6. The platform base 5 also includes an upstanding support bar 16 which, as it extends upwards provides a footrest 14 and as it extends upwards further, an armrest 15, collectively providing the patient 64 safety while standing on the platform base 5 as seen in FIG. 2.

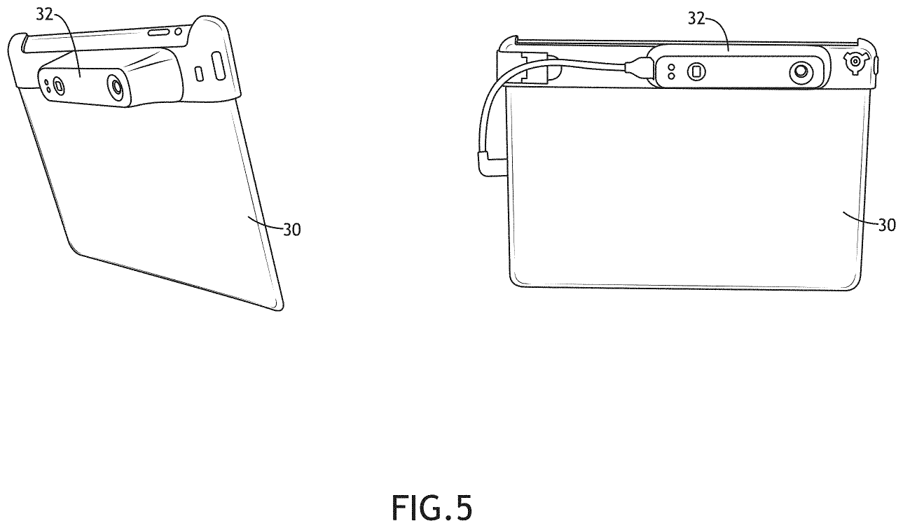

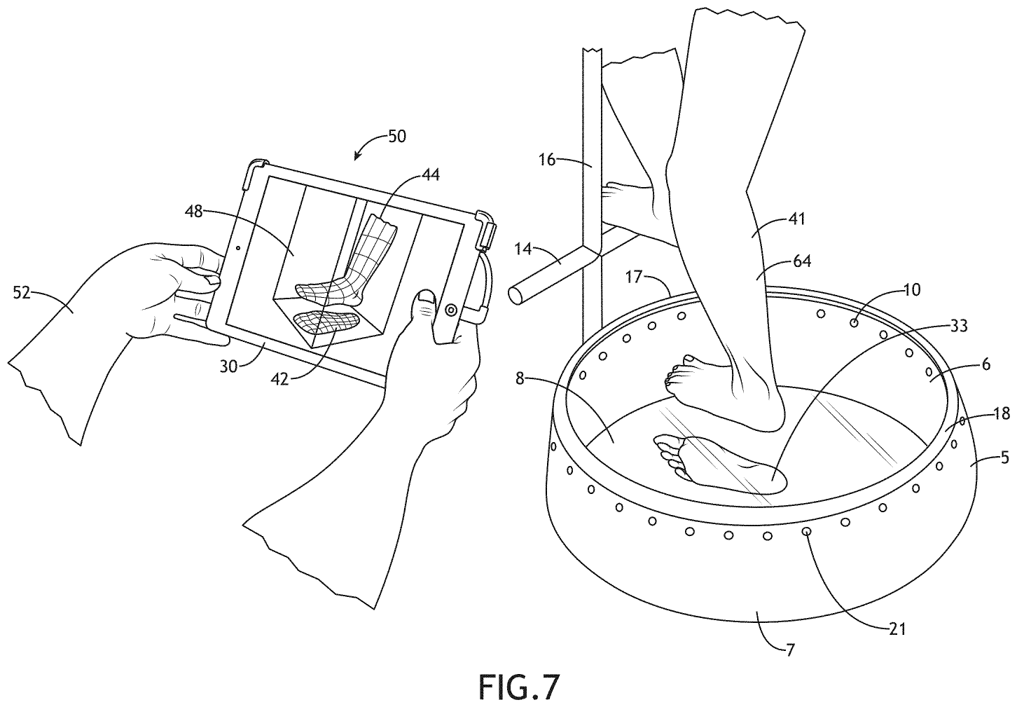

In one embodiment a guide track, which provides negative space for a guide roller 19 used to host a monopod 18 is affixed to the outer rim 17 of the platform base 5, hosts a monopod 20 used to host a three-dimensional image capturing device 22 that can travel up to 360° around the platform base 5 as seen in FIG. 4. In an exemplarily embodiment, the image-capturing device 22 is hand-held and removed from the rotational monopod 20 as seen in FIG. 7. The image capturing device 22 is a mobile computing device or tablet 30 which hosts a camera 32 with precise three-dimensional vision, like the Structure.io depth sensor or camera as seen in FIG. 5.

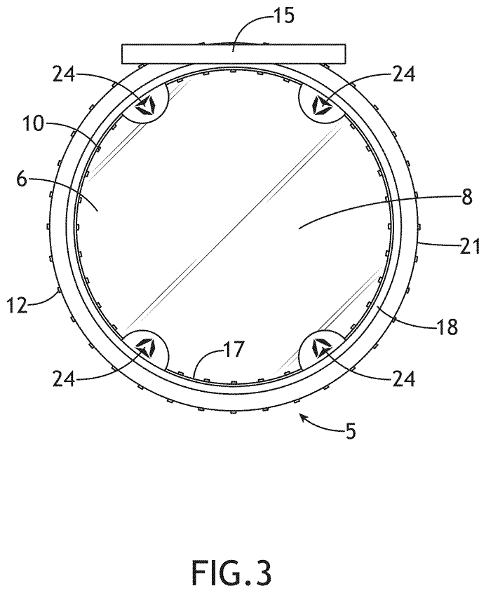

In one embodiment, interior rim lighting 10 lines the outer rim 17 of the platform base 5, and exterior rim lighting 12 lines the exterior outer rim 21 of the top of the platform base 5 creating optimal lighting for consistent three-dimensional imaging as seen in FIGS. 2 and 3.

Fiducial identification markers 24 are equally spaced and located on transparent top panel 6, as seen in FIG. 3. These fiducial identification markers 24 orient the images captured.

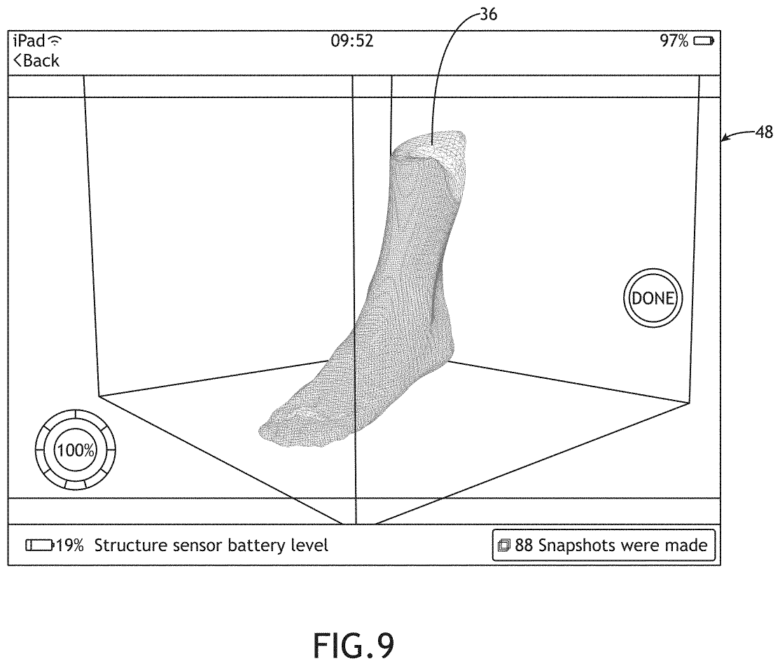

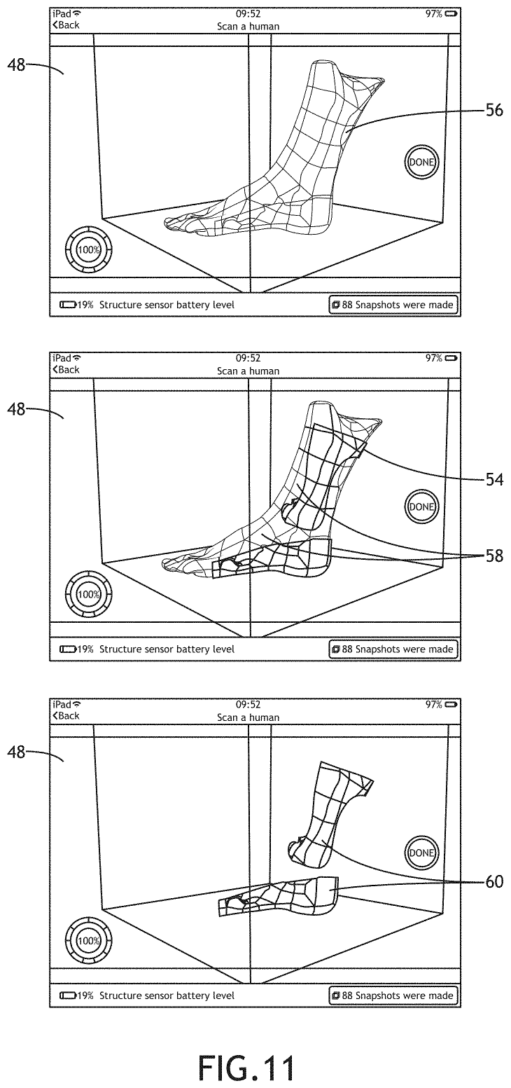

The practitioner 52 positions the patient 64 on the platform base 5. The patient 64 places the affected leg on the transparent top panel 6 and rests the non-affected leg on the footrest 14, as seen in FIGS. 6 and 7. In one embodiment, the three-dimensional imaging is initiated by launching the UI/UX 48 as shown in FIGS. 9 and 11. A flat mirror 8 affixed to the platform base 5 located below the transparent top panel 6 provides a reflection of the plantar region of the foot 33 in a weight-bearing position as well as the other anatomical portions of interest, including but not limited to the upper foot, ankle and lower leg 41. Interior rim lighting 10 lines the interior and exterior of the transparent top panel 6, creating optimal lighting for consistent three-dimensional imaging.

A tablet 30 hosting a three-dimensional image capture device 32, like the Structure.io, as seen in FIG. 5, is attached to a monopod 20 by a device holder 31, as seen in FIG. 2. The monopod 20, attached to the guide track 18 affixed to the exterior rim 21 of the transparent top panel 6, rotates around the platform base 5 and the patient 64.

Using the monopod guide, or handle 23, the practitioner 52 moves the tablet 30 hosting the digital capture device 32 around the patient 64 and begins capturing the desired images of the anatomical portion, including the reflected image of the plantar region of the foot 33, as seen in FIG. 6. Or in an exemplary embodiment a tablet 30 hosting an image capture device 32 is handheld 50 by a practitioner 52, as seen in FIG. 7

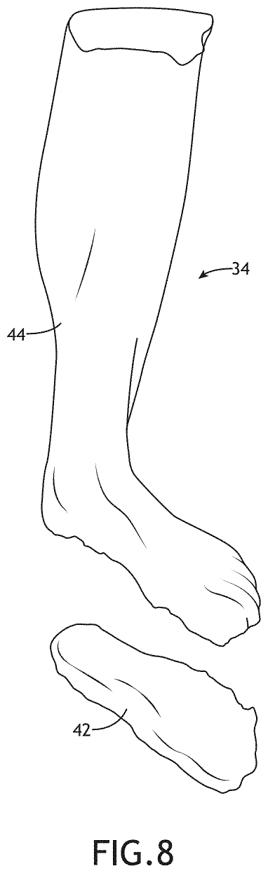

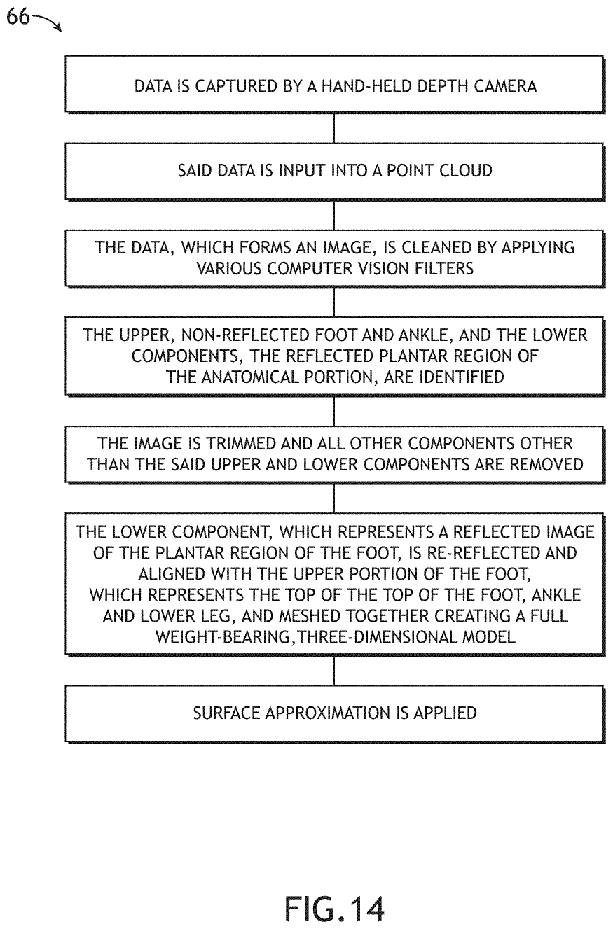

In one embodiment, an algorithm 66, as seen in FIG. 14, and collectively described herein, begins to work with the images 42 and 44 to form an anatomical model 34 by identifying the points from the plantar region 42 along with the points of the upper portion of the foot, ankle and lower leg 44 as seen in FIG. 8. The plantar images 42 are then re-reflected, aligned with the upper portion of the foot, ankle and lower leg 44 and meshed creating a full, weight-bearing, three-dimensional model 36 as seen in FIG. 9.

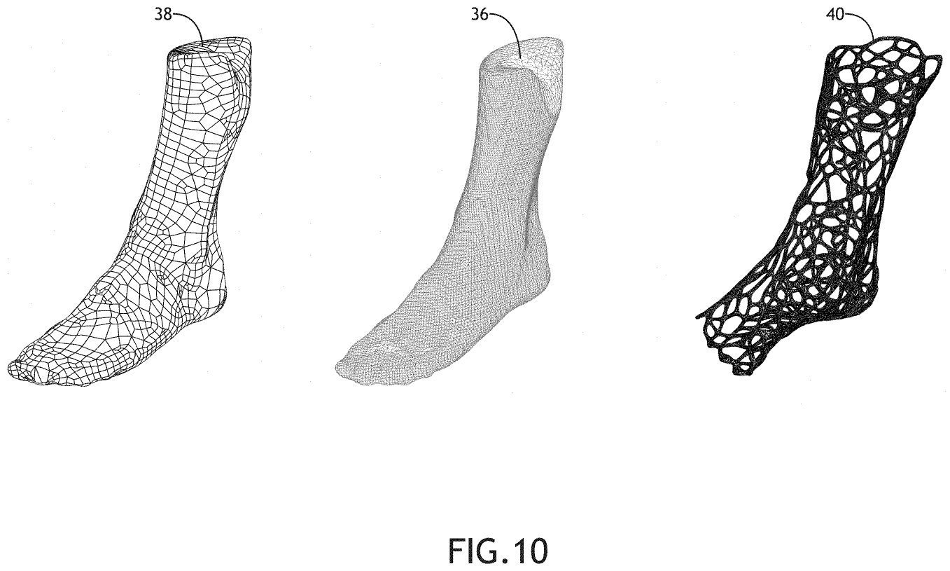

Once the full anatomical portion 34 has been captured, the algorithm 66 analyzes, trims and applies various computer-vision-filters to remove any nonmanifold vertices and edges, removing components that are not the foot, ankle or lower leg, applying a delamination filter, eroding edges, and removing vertices outside of specified volumes, among others. The algorithm 66 then cleans and isolates the data by smoothing the surfaces of the meshed image 36 in a point cloud, or a set of data points in a space generally produced by three-dimensional scanners, which measure the external surfaces of objects into a smooth, three-dimensional, customized polygonal mesh shell 38 as seen in FIG. 10 of the patient's 64 anatomical portion.

The three-dimensional polygonal mesh shell 38 is then converted into a workable CAD model 36 as seen in FIG. 10. which is used as a reference to customize the orthosis. In another embodiment, the CAD model 36 can be restructured into a three-dimensional, printable, lattice structure 40 also used as a reference to customize an orthosis, also illustrated in FIG. 10.

The practitioner 52 is then able to modify and manipulate the image within the UI/UX interface 48 to meet practitioner requirements into the orthosis. The model is then further edited for application by laying a stencil or template 54 onto the model, removing the unnecessary portions 58 of the model 36 including portions of the heel and lower leg as well as the interior section creating the customized components of the orthosis 60, as seen in FIG. 11.

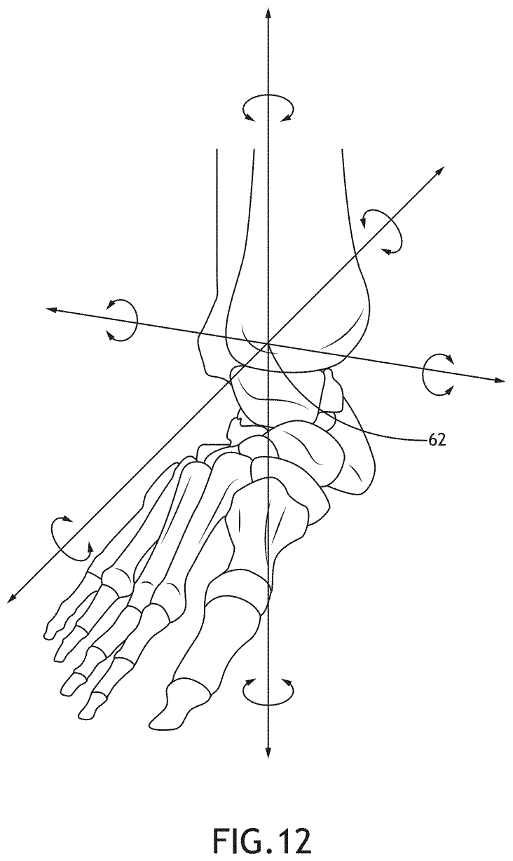

The appropriate location for a joint 28 is then determined and added based on patient-specific anatomic landmarks, as seen in FIG. 12, which are obtained from the model 36 and digitally inserted at the ankle joint line, or axis of rotation 62, as seen in FIG. 13.

The completed files are sent to a three-dimensional printer or CNC device. The fully-customized orthopedic orthosis is printed and assembled as seen in FIG. 13.

1: We claim an image-capturing platform designed for creating a three-dimensional, fully-customized, orthopedic orthoses, comprising:
a platform base comprising a frame, bottom panel, and a transparent top panel;
a mirror, located inside the said platform base, below the said transparent top panel, provides a plantar reflection of the foot, ankle, and lower leg in a weight-bearing position;
an upstanding support bar provides a footrest and an armrest;
an image-capturing device is hand-held and manually maneuvered around the anatomical portion of interest and the said platform base; and
fiducial identification markers, equally spaced and located on the said transparent top panel, located atop the said platform base, work together to form a device that creates optimal positioning to digitally capture the anatomical portion of interest in a weight-bearing position.

2: We claim a method for digitally capturing three-dimensional anatomical images for the purpose of fabricating custom, orthopedic orthoses, the method comprises:
a patient is positioned on an image-capturing platform with the affected anatomical portion of interest on a transparent top panel;
a flat mirror, affixed within a platform base, located below a transparent top panel, provides a reflection of the plantar region of the foot, ankle, and lower leg in a weight-bearing position;
an image-capturing device is hand-held and manually maneuvered around the said foot, ankle, and lower leg which allows image capturing of the desired images of the said foot, ankle, and lower leg, including the said reflected image of the plantar region of the foot;
three-dimensional image capturing is initiated by the practitioner launching a UI/UX platform, capturing images of the said foot, ankle, and lower leg in a weight-bearing position on the said platform base, collectively obtaining multiple, three-dimensional images from a plurality of angles and points;
an algorithm begins to work with the said images captured to clean and form a merged, anatomical model by identifying the points, from a point cloud, of the said plantar region along with the points of the upper portion of the said foot, ankle, and lower leg;
the said plantar images are then re-reflected, aligned, and meshed with the points of the said upper components of the said foot, ankle, and lower leg creating a full weight-bearing, three-dimensional model;
the said algorithm analyzes, trims, and removes any residual reflections, or unwanted visual elements, by smoothing the surfaces of the said meshed three-dimensional model in the point cloud into a smooth, three-dimensional, customized, polygonal mesh shell of the foot, ankle, and lower leg;
the said three-dimensional, polygonal mesh shell is then converted into a workable CAD model used as a reference to customize an orthosis;
the said CAD model is modified and manipulated based on specified practitioner requirements within the said UI/UX;
the said CAD model is further edited for application by laying a stencil, or template, onto the said model, removing the unnecessary portions of the model, creating separate customized components,
the said CAD model is pulled from the said point cloud and uploaded into a file;
materials are specified, and completed files are sent to a three-dimensional printer or CNC device; and
a fully-customized, orthopedic orthosis is printed and assembled.

3: We claim a device of claim 1, wherein the said image-capturing platform hosts a guide track, affixed to the said platform base, which provides negative space for a guide roller, which hosts a monopod that guides an image-capturing device around the said image-capturing platform.

4: We claim a device of claim 1, wherein the image-capturing device is a mobile computing device or tablet which hosts a camera with precise three-dimensional vision or a depth sensor.

5: We claim a device of claim 1, wherein exterior and interior lighting lines the said platform base creating optimal lighting for consistent imaging.

6. We claim a device of claim 1, wherein the platform base includes at least four fiducial identification markers.

7. We claim a device of claim 1, wherein the platform base comprises a circular, oval, or square like frame.

8. We claim a device of claim 1, wherein the transparent top panel is made of plexiglass, glass, or a similar transparent material.

9. We claim a method of claim 2, wherein:
the joint line, or axis of rotation, is identified based on patient-specific anatomic landmarks; and
a cam is inserted at the said joint line, or axis of rotation.

10. We claim a method of claim 2, wherein the cam, inserted at the said joint line, or axis of rotation, is made of polyurethane, or a similar flexible material.

11. We claim a method of claim 2, wherein the CAD model can be restructured into a three-dimensional, printable, lattice structure, used as a reference to customize an orthosis.

12. We claim a method of claim 2, wherein the said image-capturing platform hosts a guide track, affixed to the said platform base, which provides negative space for a guide roller, which hosts a monopod that guides the said image-capturing device around the said image-capturing platform.

13. We claim a method of claim 2, wherein the removed portions of the model include the negative space from the stencil, or template, included but not limited to the heel and lower leg, as well as the interior section of the model.

14. We claim a method of claim 2, wherein the fully-customized orthopedic orthosis is made of polypropylene, nylon, or a similar material.
